(19) United States
(12) Patent Application Publication
(10) Pub. No.: US 2008/0233209 A1
(43) Pub. Date: Sep. 25, 2008

(54) Composition and method of use for the treatment of metabolic syndrome and inflammation

(75) Inventor: Nancy E. Fogg-Johnson, Villanova, PA (US)

Correspondence Address: KRAMER & AMADO, P.C., 1725 DUKE STREET, SUITE 240, ALEXANDRIA, VA, 22314, US

(73) Assignee: POLIFENOLES NATURALES, SL, Ingenio (ES)

(21) Appl. No.: 12/076,831

(22) Filed: Mar. 24, 2008

Related U.S. Application Data
(60) Provisional application No. 60/919,432, filed on Mar. 22, 2007.

(51) Int. Cl. A61K 33/24; A61K 31/336; A61K 31/381; A61K 36/54

(52) U.S. Cl. 424/655; 514/475; 424/739; 514/440

(57) ABSTRACT

The present invention provides a method and composition for preventing, treating or managing Metabolic Syndrome. The composition contains brown marine vegetable extract containing an effective amount of fucoxanthin which is administered for preventing, treating or managing Metabolic Syndrome.

### CROSS-REFERENCE TO RELATED APPLICATION

This application is related to provisional application Ser. No. 60/919,432, filed Mar. 22, 2007, which is incorporated by reference, and claims the benefit of its earlier filing date under 35 USC Section 119(e).

### FIELD OF THE INVENTION

This invention relates to a composition and method for treating metabolic syndrome and inflammation, and in particular, the use of an effective amount of an extract of a brown marine vegetable, particularly fucoxanthin in a composition.

### BACKGROUND

Metabolic Syndrome

Metabolic syndrome (MetS) is a cluster of metabolic abnormalities, including abdominal or visceral obesity, glucose intolerance, hypertension and dyslipidaemia, that is associated with an increased risk of cardiovascular disease and/or vascular events that is increasing at epidemic rates in westernized countries. The collection of cardiovascular disease (CVD) risk factors—including hypertension and dyslipidemia—and type 2 diabetes, and their association with insulin resistance led investigators to propose the recognition of a distinct condition called “the metabolic syndrome,” which has been defined by reputable organizations and assigned its own code in the World Health Organization's (WHO) ICD-9. The WHO criteria for a diagnosis of metabolic syndrome require the presence of diabetes mellitus, impaired glucose tolerance, impaired fasting glucose or insulin resistance, and further includes an analysis of blood pressure, dyslipidemia; central obesity, and microalbuminuria. The liver includes one of the primary insulin-sensitive tissues, and insulin resistance has an significant role in both metabolic syndrome, as well as the development of non-alcoholic fatty liver disease (NAFLD). NAFLD, along with insulin resistance, obesity, diabetes, dyslipidemia, and nonalcoholic fatty liver, is an underlying component of metabolic syndrome.

The National Cholesterol Education Program (NCEP) and particularly, the International Diabetes Foundation (IDF), have taken the position that obesity (especially abdominal obesity) is a dominant factor behind the multiplication of risk factors in MetS. According to the NCEP, the onset of obesity elicits a clustering of risk factors in persons who are metabolically susceptible. Metabolic susceptibility has many contributing factors, including genetic forms of insulin resistance, increased abdominal or visceral fat, ethnic and racial influences, physical inactivity, advancing age, endocrine dysfunction, and genetic diversity.

Visceral obesity is the accumulation of adipose tissue inside the abdominal cavity, in particular at omental and mesenteric regions, which are drained by the portal vein and therefore have direct access to the liver. Emerging information links obesity and basal (i.e., constitutive) inflammation with metabolic syndrome. The term “the metabolic syndrome” is thus typically used as shorthand notation to indicate a clustering of CVD factors of metabolic origin.

There are 2 primary schools of thought about the best therapeutic strategy for patients with the metabolic syndrome. One view holds that each of the metabolic risk factors should be singled out and treated separately. The other view holds that greater emphasis should be given to implementing therapies that will reduce all of the risk factors simultaneously. The latter approach emphasizes lifestyle therapies (specifically weight reduction and increased exercise), which target all of the risk factors. This approach is also the foundation of other therapies for targeting multiple risk factors together by striking at the underlying causes, as in the identification and development of substances and treatment regimens to promote weight reduction and to reduce insulin resistance. There is a great need for safe effective weight-reduction substances to aid in prevention, treatment and management of MetS, particularly the main initiating factor, obesity.

To fully understand the significance of the metabolic syndrome, it is necessary to understand that the condition begins insidiously with abdominal obesity and/or insulin resistance and progresses over time (some individuals who are particularly susceptible to the syndrome will have an accelerated progression; risk factors can develop as a result of only mild abdominal obesity).

The core risk factors of the metabolic syndrome are atherogenic dyslipidaemia, elevated blood pressure, elevated plasma glucose, a prothrombotic state and a pro-inflammatory state. According to current views, there are two major underlying causes: obesity (especially visceral or abdominal obesity) and insulin resistance.

Progression of the metabolic syndrome begins with obesity and/or insulin resistance. In the early stages, the metabolic risk factors are often only marginally increased, but with time, particularly when obesity increases and other exacerbating factors become involved, the risk factors increase considerably. Excess body fat, particularly when present in the upper body as abdominal or visceral adiposity, is one contributing cause of the metabolic syndrome. In abdominally obese individuals with the metabolic syndrome, weight reduction, which is unique among available therapeutic strategies, will reduce all of the metabolic risk factors. This documented efficacy accounts largely for the great interest in the development of safe non-drug, natural and dietary means to treat obesity.

A weight-loss agent that is effective and can be tolerated for long periods almost certainly would be beneficial for the syndrome as a whole. An ideal substance for weight loss would be a natural component of the diet or fractions of dietary components, which when combined, provide the unexpected benefit of weight loss. Provision of such a substance or combination of substances would be a highly attractive solution to the increasing incidence of obesity, metabolic syndrome and inflammation.

Obesity, Inflammation, Insulin Resistance and the Metabolic Syndrome

Increasingly, insulin resistance has been recognized as an integral feature of the metabolic syndrome as well as any other features such as obesity, glucose intolerance, hypertriglyceridemia, low HDL cholesterol, hypertension, and accelerated atherosclerosis. Insulin regulates the uptake, oxidation and storage of fuel in insulin-sensitive tissues, such as the liver, skeletal muscle, adipose tissue, and also macrophages. Obesity, and in particular visceral obesity (which is the accumulation of adipose tissue inside the abdominal cavity), is associated with resistance to the effects of insulin (insulin resistance) on peripheral glucose and fatty-acid utilization, often leading to type 2 diabetes mellitus.

With the recent trend for individuals to be more obese, a large increase in the prevalence of insulin resistance in westernized countries, as well as in developing countries, is occurring. The incidence is spreading from the adult population to children, due primarily to increasing incidence of obesity among younger populations.

It is now well-accepted and clear that obesity is associated with a state of chronic low-level inflammation which has an important role in the pathogenesis of insulin resistance and type 2 diabetes mellitus. Population studies show a strong correlation between the levels of pro-inflammatory biomarkers, such as C-reactive protein (CRP), interleukin-6 (IL-6) and tumor-necrosis factor (TNF), and perturbations in glucose homeostasis, obesity and atherosclerosis. Overproduction of TNF-a in adipose tissue is an important feature of obesity and contributes significantly to insulin resistance. Obesity is characterized by a broad inflammatory response in which many inflammatory mediators exhibit patterns of expression and/or impact insulin action in a manner similar to that of TNF-A during obesity. Such a characterized response has been documented in animals ranging from mice and cats to humans. Lipids themselves also participate in the coordinated regulation of inflammation and metabolism. Elevated plasma lipid levels are characteristic of obesity, infection, and other inflammatory states. For example, hyperlipidemia in obesity is responsible in part for inducing peripheral tissue insulin resistance and dyslipidemia and further contributes to the development of atherosclerosis.

Obesity and the associated chronic inflammatory response, which is characterized by abnormal cytokine production, increases synthesis of acute-phase reactants, such as C-reactive protein (CRP), and the activation of pro-inflammatory signaling. Chronic inflammation in fat plays a crucial role in the development of obesity-related insulin resistance and, obesity-related insulin resistance is, at least in part, a chronic inflammatory disease initiated in adipose tissue. Adipose tissue has been shown to be a source of many products that in one way or another can worsen the metabolic syndrome, and abnormalities in the release of these products have been amply demonstrated in obese persons.

Adipose tissue in obese persons produces multiple adipokines, soluble cytokines produced mainly by adipose tissue, which contribute to development of the metabolic syndrome. Adipose tissue releases non-esterified fatty acids (NEFA) after lipolysis of triglyceride. Excess NEFA release in obesity overloads muscle, liver and pancreatic B-cells with lipids. This ectopic lipid accumulation adds significantly to insulin resistance, atherogenic dyslipidaemia and hyperinsulinemia.

Systemic chronic inflammation has an important role in the pathogenesis of obesity related insulin resistance as well. Biomarkers of inflammation, such as TNF, IL-6 and CRP, are present at increased concentrations in individuals who are insulin resistant and obese, and these biomarkers can predict the development of type 2 diabetes mellitus and cardiovascular diseases. Human studies show increased TNF expression in the adipose tissue of individuals who were obese, and decreased TNF expression after weight loss.

An additional reason emphasizing the importance of maintaining a healthy weight is the emerging paradigm that metabolic imbalance leads to immune imbalance, with starvation and immunosuppression on one end of the spectrum and obesity and inflammatory diseases on the other end. It now appears that, in most obese patients, obesity is associated with a low-grade inflammation of white adipose tissue (WAT) resulting from chronic activation of the innate immune system which can subsequently lead to insulin resistance, impaired glucose tolerance and even diabetes. WAT is the physiological site of energy storage as lipids. In addition, it has been more recently recognized as an active participant in numerous physiological and pathophysiological processes. In obesity, WAT is characterized by an increased production and secretion of a wide range of inflammatory molecules including TNF-α and interleukin-6 (IL-6), which may have local effects on WAT physiology but also systemic effects on other organs. Recent data indicate that obese WAT is infiltrated by macrophages, which may be a major source of locally-produced pro-inflammatory cytokines. Interestingly, weight loss is associated with a reduction in the macrophage infiltration of WAT and an improvement of the inflammatory profile of gene expression. Several factors derived not only from adipocytes but also from infiltrated macrophages probably contribute to the pathogenesis of insulin resistance. Most of them are overproduced during obesity, including leptin, TNF-α, IL-6 and resistin. Conversely, expression and plasma levels of adiponectin, an insulin-sensitizing effector, are down-regulated during obesity.

Leptin could modulate TNF-α production and macrophage activation. TNF-α has been overproduced in adipose tissue of several rodent models of obesity and plays a significant role in the pathogenesis of insulin resistance in these species. However, its actual involvement in glucose metabolism disorders in humans remains controversial. IL-6 production by human adipose tissue also increases during obesity. Further, it may induce hepatic CRP synthesis and may promote the onset of cardiovascular complications. Both TNF-α and IL-6 can alter insulin sensitivity by triggering different key steps in the insulin signaling pathway. In rodents, resistin can induce insulin resistance, while its implication in the control of insulin sensitivity is still a matter of debate in humans. Adiponectin is highly expressed in WAT, and circulating adiponectin levels are decreased in subjects with obesity-related insulin resistance, type-2 diabetes and coronary heart disease. Adiponectin inhibits liver neoglucogenesis and promotes fatty acid oxidation in skeletal muscle. In addition, adiponectin counteracts the pro-inflammatory effects of TNF-α on the arterial wall and may very well protect against the development of arteriosclerosis.

In obesity, the pro-inflammatory effects of cytokines through intracellular signaling pathways involve the NF-κB and JUN N-terminal kinase (JNK) systems. Genetic or pharmacological manipulations of these effectors of the inflammatory response have been shown to modulate insulin sensitivity in different animal models. In humans, it has been suggested that the improved glucose tolerance observed in the presence of thiazolidinediones or statins is likely related to their anti-inflammatory properties. Thus, it can be considered that obesity corresponds to a sub-clinical inflammatory condition that promotes the production of pro-inflammatory factors involved in the pathogenesis of insulin resistance. While thiazolidinediones or statins classes of drugs may have salutatory effects on some aspects of MetS, each of those classes of drugs is associated with potential undesirable side effects. Thus, there is a need for effective, safe dietary treatments that can facilitate weight loss and management of other aspects of MetS, such as inflammation and imbalance in pro- and anti-inflammatory cytokines and adipocytokines.

Even in the absence of obesity, infusion of animals with inflammatory cytokines or lipids can cause insulin resistance. Additionally, humans with some other chronic inflammatory conditions are at increased risk for diabetes. Finally, removal of inflammatory mediators or pathway components, such as TNF-α, JNK, and IKK, protects against insulin resistance in obese mouse models, and treatment of humans with drugs that target these pathways, such as salicylates, improves insulin sensitivity. Thus, the available evidence strongly suggests that type 2-diabetes is an inflammatory disease and that inflammation is a primary cause of obesity-linked insulin resistance, hyperglycemia, and hyperlipidemia rather than merely a consequence.

It seems likely that the inflammatory response is initiated in the adipocytes themselves, as they are the first cells affected by the development of obesity. Further, neighboring cells may potentially be affected by adipose growth. One mechanism that appears to be of central importance is the activation of inflammatory pathways by Endoplasmic Reticulum (ER) stress. Obesity generates conditions that increase the demand on the ER. This is particularly the case for adipose tissue, which undergoes severe changes in tissue architecture, increases in protein and lipid synthesis, and perturbations in intracellular nutrient and energy fluxes. In both cultured cells and whole animals, ER stress leads to activation of JNK and thus contributes to insulin resistance. Interestingly, ER stress also activates IKK and thus may represent a common mechanism for the activation of these 2 important signaling pathways.

A second mechanism that may be relevant in the initiation of inflammation in obesity is oxidative stress. Due to increased delivery of glucose to adipose tissue, endothelial cells in the fat pad may take up increasing amounts of glucose through their constitutive glucose transporters. Increased glucose uptake by endothelial cells in hyperglycemic conditions causes excess production of ROS in mitochondria, which inflicts oxidative damage and activates inflammatory signaling cascades inside endothelial cells. Endothelial injury in the adipose tissue might attract inflammatory cells such as macrophages to this site and further exacerbate the local inflammation. Hyperglycemia also stimulates ROS production in adipocytes, which leads to increased production of proinflammatory cytokines.

Moreover, obesity alters adipose tissue metabolic and endocrine function and leads to an increased release of fatty acids, hormones, and proinflammatory molecules that contribute to obesity associated complications. Adiposity, which is the fraction of total body mass comprised of neutral lipid stored in adipose tissue, is closely correlated with important physiological parameters such as blood pressure, systemic insulin sensitivity, and serum triglyceride and leptin concentrations. Increased adipocyte volume and number are positively correlated with leptin production, and leptin is an important regulator of energy intake and storage, insulin sensitivity, and metabolic rate. Leptin signaling has also been implicated in the pathogenesis of arterial thrombosis. Adiposity is negatively correlated with production of adiponectin (also known as ACRP30), a hormone that decreases hepatic gluconeogenesis and increases lipid oxidation in muscle. In addition, strong, positive correlations exist between degree of adiposity and several obesity-associated disorders such as hypertension, dyslipidemia, and glucose intolerance. Visceral fat mass is more closely correlated with obesity-associated pathology than overall adiposity. Obesity in humans is an independent risk factor for myocardial infarction, stroke, type-2 diabetes mellitus, and certain cancers.

The altered production of proinflammatory molecules (so-called “adipokines”) by adipose tissue has been implicated in the metabolic complications of obesity. Compared with adipose tissue of lean individuals, adipose tissue of the obese expresses increased amounts of proinflammatory proteins such as TNF-α, IL-6, iNOS (also known as NOS2), TGF-β1, C-reactive protein (CRP), soluble ICAM, and monocyte chemotactic protein-1 (MCP-1), and procoagulant proteins such as plasminogen activator inhibitor type-1 (PAI-1), tissue factor, and factor VII. Proinflammatory molecules have direct effects on cellular metabolism. For example, TNF-α directly decreases insulin sensitivity and increases lipolysis in adipocytes. IL-6 leads to hypertriglyceridemia in vivo by stimulating lipolysis and hepatic triglyceride secretion.

Obesity and Immune Function

The incidence of obesity and its associated disorders is increasing markedly worldwide. Obesity predisposes individuals to an increased risk of developing many diseases, including atherosclerosis, diabetes, nonalcoholic fatty liver disease (NAFLD), certain cancers and some immune-mediated disorders, such as asthma. In addition to these associations between obesity and disease, research in the past few years has identified important pathways that link metabolism with the immune system and vice versa. Many of these interactions between the metabolic and immune systems seem to be orchestrated by a complex network of soluble mediators derived from immune cells and adipocytes (fat cells).

Obesity and the associated metabolic pathologies are the most common and detrimental metabolic diseases, affecting over 50% of the adult population. These conditions are associated with a chronic inflammatory response characterized by abnormal cytokine production, increased acute-phase reactants, and activation of inflammatory signaling pathways. This association is not an inconsequential one, at least in experimental models, and is causally linked to either obesity itself or closely linked diseases such as insulin resistance, type 2 diabetes, and cardiovascular disease.

A very interesting feature of the inflammatory response that emerges in the presence of obesity is that it appears to be triggered, and to reside predominantly, in adipose tissue, although other metabolically critical sites may also be involved during the course of the disease. Obese adipose tissue is characterized by inflammation and progressive infiltration by macrophages as obesity develops. Changes in adipocyte and fat pad size lead to physical changes in the surrounding area and modifications of the paracrine function of the adipocyte. For example, in obesity, adipocytes begin to secrete low levels of TNF-α, which can stimulate preadipocytes to produce monocyte chemoattractant protein-1 (MCP-1). Similarly, endothelial cells also secrete MCP-1 in response to cytokines. Thus, either preadipocytes or endothelial cells could be responsible for attracting macrophages to adipose tissue. Increased secretion of leptin (and/or decreased production of adiponectin) by adipocytes may also contribute to macrophage accumulation by stimulating transport of macrophages to adipose tissue and promoting adhesion of macrophages to endothelial cells, respectively. It is conceivable, also, that physical damage to the endothelium, caused either by sheer size changes and crowding or oxidative damage resulting from an increasingly lipolytic environment, could also play a role in macrophage recruitment, similar to that seen in atherosclerosis. Whatever the initial stimulus to recruit macrophages into adipose tissue is, once these cells are present and active, they, along with adipocytes and other cell types, could perpetuate a vicious cycle of macrophage recruitment, production of inflammatory cytokines, and impairment of adipocyte function.

In mammals, adipose tissue occurs in two forms: white adipose tissue and brown adipose tissue. Most adipose tissue in mammals is white adipose tissue and this is thought to be the site of energy storage. By contrast, brown adipose tissue is found mainly in human neonates and is important for the regulation of body temperature through non-shivering thermogenesis. In addition to adipocytes, which are the most abundant cell type in white adipose tissue, adipose tissue also contains pre-adipocytes (which are adipocytes that have not yet been loaded with lipids), endothelial cells, fibroblasts, leukocytes and, most importantly, macrophages. These macrophages are bone-marrow derived and the number of these cells present in white adipose tissue correlates directly with obesity.

Adipose tissue is no longer considered to be an inert tissue functioning solely as an energy store, but is emerging as an important factor in the regulation of many pathological processes. Various products of adipose tissue have been characterized, and some of the soluble factors produced by this tissue are known as adipocytokines.

The term adipocytokine is used to describe certain cytokines that are mainly produced by adipose tissue, although it is important to note that they are not all exclusively derived from this organ. Adiponectin, leptin, resistin and visfatin are adipocytokines and are thought to provide an important link between obesity, insulin resistance and related inflammatory disorders. Adiponectin and leptin are the most abundant adipocytokines produced by adipocytes. Various other products of adipose tissue that have been characterized include: certain cytokines, such as tumour-necrosis factor (TNF), interleukin-6 (IL-6), IL-1 and CC-chemokine ligand 2 (CCL2; also known as MCP 1); mediators of the clotting process, such as plasminogen-activator inhibitor type 1; and certain complement factors. These products have well-known roles in the immune system, and although some of them are also produced by adipocytes, they are not normally considered to be adipocytokines; nonetheless, they have important roles at the interface between the immune and metabolic systems.

Serum levels of adiponectin are markedly decreased in individuals with visceral obesity and states of insulin resistance, such as non-alcoholic fatty liver disease, atherosclerosis and type 2 diabetes mellitus, and adiponectin levels correlate inversely with insulin resistance. It has been suggested recently that the ratio, and not the absolute amounts, of high-molecular-weight and low-molecular-weight adiponectin in the serum might be crucial in determining insulin sensitivity.

Insulin resistance may be partly precipitated or accelerated by an acute-phase reaction as part of the innate immune response, in which large amounts of pro-inflammatory mediators and insufficient amounts of anti-inflammatory mediators, such as adiponectin, are released from adipose tissue. TNF suppresses the transcription of adiponectin in an adipocyte cell line, which might explain the lower levels of serum adiponectin in individuals who are obese. Expression of adiponectin is also regulated by other pro-inflammatory mediators such as IL-6, which suppresses adiponectin transcription and translation in an adipocyte cell line. Weight loss is a potent inducer of adiponectin synthesis, as is activation of peroxisome proliferator-activated receptor gamma (PPAR-g). Peroxisome-proliferator activated receptor-gamma, which is a nuclear receptor that is a master transcriptional regulator of metabolism and fat-cell formation. The activity of PPAR-gamma can be modulated by the direct binding of small molecules, such as thiazolidinediones, drugs used in treatment of type-2 diabetes. PPAR-g has anti-inflammatory properties by limiting the availability of limited cofactors or blocking promoters of pro-inflammatory genes.

Circulating levels of adiponectin, however, are also affected by many other factors including gender, age and lifestyle. In obese animals, treatment with adiponectin decreases hyperglycemia and levels of free fatty acids in the plasma, and improves insulin sensitivity. Specific PPAR-gamma agonists, such as thiazolidinediones, improve insulin sensitivity by mechanisms that are largely unknown. Circulating levels of adiponectin are significantly upregulated in vivo after activation of PPAR-g. Mice lacking adiponectin not only have decreased hepatic insulin sensitivity but also have reduced responsiveness to PPAR-g agonists, which indicates that adiponectin is an important contributor to PPAR-g-mediated improvements in insulin sensitivity. Adiponectin stimulates B-oxidation in rat hepatocytes and down regulates expression of sterol-regulatory-element-binding protein 1C (SREBP1C), which is the main transcription factor regulating expression of genes encoding mediators of lipid synthesis. Sustained peripheral, ectopic expression of adiponectin decreases the development of diet-induced obesity and improves insulin sensitivity. Together, these studies strongly support a major role for adiponectin in regulating insulin sensitivity.

As important mediator in the regulation of insulin resistance, adiponectin can suppress inflammation in various animal models. This adipocytokine also has a crucial role in suppressing macrophage activity, not only in adipose tissue but also in other tissues such as the liver. Decreased synthesis of adiponectin, as is observed in individuals who are obese, might lead to dysregulation of the controls that inhibit the production of pro-inflammatory cytokines, thereby leading to the production of increased amounts of pro-inflammatory mediators. One of the main challenges in understanding the physiology of this adipocytokine will be to understand why circulating levels decrease with the onset of obesity. Also of great interest is how this decrease might affect the cytokine-adipocytokine milieu, resulting in an overwhelmingly pro-inflammatory state.

Leptin is a proinflammatory cytokine that is elevated in circulation of obese individuals. The role of leptin in modulating the immune response and inflammation has become increasingly evident. In addition to regulating neuroendocrine function, energy homeostasis, haematopoiesis and angiogenesis, this adipocytokine is an important mediator of immune-mediated diseases and inflammatory processes. Similar to adiponectin, leptin is produced mainly by adipocytes. However, unlike adiponectin, leptin is considered to be a pro-inflammatory cytokine and it has structural similarity to other pro-inflammatory cytokines such as IL-6, IL-12 and granulocyte colony stimulating factor. The main function of leptin is control of appetite.

Serum levels of leptin reflect the amount of energy stored in the adipose tissue and are proportional to overall adipose mass in humans. Serum levels are 2-3 times higher in women than in men, even when adjusted for age and body-mass index (BMI). In animal models, expression of leptin is increased in conditions that are associated with the release of pro-inflammatory cytokines, as induced during acute inflammatory conditions such as sepsis. An increase in leptin levels and a decrease in expression of mRNA encoding the full-length isoform b receptor (OBRb, which is one of at least six alternatively spliced isoforms, each of which has a cytoplasmic domain of a different length) has been observed in diet-induced obese rats. In addition to adipose tissue, leptin is produced by several other tissues, including placenta, bone marrow, stomach, muscle and perhaps the brain. Therefore, pro-inflammatory mediators and obesity seem to be the main factors responsible for increased leptin synthesis. Despite this evidence of a role for leptin in immune responses in vitro and in mouse models, it is currently unclear whether leptin influences immune responses in humans. Nevertheless, it is clear that leptin has proinflammatory effects.

Resistin (also known as FIZZ3), which is a 114-amino-acid polypeptide, was originally shown to induce insulin resistance in mice. It belongs to a family of cysteine-rich proteins, also known as resistin-like molecules (RELMs) that have been implicated in the regulation of inflammatory processes. Resistin was shown to circulate in two distinct forms: a more prevalent high-molecular-weight hexamer and a substantially more bioactive, but less prevalent, low-molecular-weight complex. An mRNA encoding resistin can be found in mice and humans in various tissues, including adipose tissue, the hypothalamus, adrenal gland, spleen, skeletal muscle, pancreas and gastrointestinal tract. Although resistin protein synthesis in mice seems to be restricted to adipocytes, in humans, adipocytes, muscle, pancreatic cells and mononuclear cells such as macrophages can synthesize this protein. Expression levels of the gene encoding resistin have been shown to be higher in human peripheral-blood mononuclear cells (PBMCs) than in adipocytes; however, comparative protein data are not available. So, it still remains to be shown which cell type in humans is mainly responsible for systemic production and for the high circulating levels of resistin. In human PBMCs, expression of resistin mRNA is markedly increased by the pro-inflammatory cytokines IL-1, IL-6 and TNF, and by LPS, whereas IFN-gamma and leptin had no effect.

Other important adipokines produced in excess with obesity are plasminogen-activator inhibitor 1(PAI1), inflammatory cytokines (tumour-necrosis factor-a (TNFa), interleukin-6 (IL-6)) and others. These seem to have a role in several metabolic risk factors, including a prothrombotic state, a pro-inflammatory state, and insulin resistance. Obese persons exhibit low adipose-tissue release of adiponectin, which has been implicated in causation of insulin resistance and fatty liver. These adipokines promote vascular dysfunction and atherogenesis either indirectly though metabolic risk factors or by direct action on the arterial wall.

Non-Alcoholic Fatty Liver Disease (NAFLD) and Metabolic Syndrome

The accumulation of TGs in the liver is a primary metabolic factor that contributes to the development of non-alcoholic fatty liver disease (NAFLD) and is a major factor in the development of insulin resistance and obesity. The term NAFLD refers to a spectrum of hepatic pathology that resembles alcoholic liver disease, but appears in individuals who have low or negligible alcohol consumption. In recent years, the condition has received considerable attention, primarily because of a better understanding of its involvement in the development of insulin resistance and obesity. The reported prevalence of NAFLD among the general population is approximately 9% in Western countries and 1.2% in Japan. However, among obese subjects living in Western countries, the prevalence of NAFLD ranges from 23-31 percent. The relationship between NAFLD and obesity has been firmly established. Most patients with NAFLD are overweight, and have some degree of insulin resistance. NAFLD is one of the main forms of chronic liver disease and is believed to be the most common pathology behind the hepatic component of metabolic syndrome, whose main features include obesity, hyperinsulinemia, peripheral insulin resistance, dyslipidemia, and hypertension. The severity of liver fat accumulation positively correlates with visceral fat content and insulin resistance in both obese and non-obese subjects, suggesting that hepatic fat infiltration in NAFLD may be influenced by visceral fat accumulation regardless of body mass index. Gender also plays an important role in the development of NAFLD because it is more prevalent among women than men, although the mechanism is not presently known. NAFLD is seen most frequently in females who are morbidly obese, have had jejunal bypass surgery and elevated levels of plasma aspartate aminotransferase (AST) and alanine aminotransferase (ALT).

NAFLD is emerging as a component of the metabolic syndrome. Although it is not known whether markers of NAFLD, including elevated concentrations of aspartate aminotransferase (AST), alanine aminotransferase (ALT), and g-glutamyltransferase (GGT), predict the development of metabolic syndrome, all of the factors associated with metabolic syndrome are interrelated. Obesity and lack of exercise tend to lead to insulin resistance. Insulin resistance has a negative effect on lipid production, increasing VLDL (very low-density lipoprotein), LDL and triglyceride levels in the bloodstream and decreasing HDL (high-density lipoprotein). This can lead to fatty plaque deposits in the arteries enhancing the risks for cardiovascular disease, blood clots, and strokes. Excess insulin increases renal sodium retention, which increases blood pressure and can lead to hypertension.

NAFLD is the major cause of elevation of ALT and it is in fact considered the hepatic manifestation of metabolic syndrome. NAFLD appears to be most strongly associated with obesity. Several studies have shown that variation in serum GGT in populations is associated with risk of death or development of cardiovascular disease, type 2 diabetes, stroke, or hypertension. Highly significant correlations have been found between GGT and body mass index, serum lipids, lipoproteins, glucose, insulin, and blood pressure. Results have also indicated that serum GGT may be an important predictor for developing metabolic syndrome and type 2 diabetes mellitus.

Type 2 Diabetes Mellitus

Type-2 diabetes mellitus is a disorder of glucose homeostasis that is characterized by inappropriately increased blood-glucose levels and resistance of tissues to the action of insulin. Recent studies indicate that inflammation in adipose tissue, liver and muscle contributes to the insulin-resistant state that is characteristic of type 2 diabetes mellitus, and that the anti-diabetic actions of peroxisome-proliferator activated receptor-g (PPAR-g) agonists result, in part, from their anti-inflammatory effects in these tissues.

Dietary Solutions to MetS and Inflammation

Large epidemiological and clinical studies provide convincing evidence for the health promoting effects of natural carotenoids. Positive effects of natural carotenoids on human health are primarily attributed to their pro-vitamin and antioxidant activity. Natural carotenoids b-carotene, lycopene, lutein, astaxanthin and fucoxanthin are well known for their anti-cancer and superior free radicals scavenging properties that are common among all carotenoids. However, recent research has revealed that, in addition to common antioxidant and anti-cancer activity, some carotenoids possess much more specific and unique pharmacological effects. Fucoxanthin, a carotenoid specific to brown marine vegetables, has been recently reported to exhibit anti-obesity and thermogenic effects. It was demonstrated that fucoxanthin upregulates the expression of uncoupling protein UCP1 gene in white adipose tissue (WAT), thus contributing to reduction of visceral fat. Fucoxanthin reduced WAT in wistar rats and obese KK-Ay mice and caused a significant reduction of body weight in the KK-Ay mice. It was suggested that fucoxanthin-induced UCP1 expression in WAT stimulates oxidation of fatty acid. It was further suggested that this effect is specific to fucoxanthin, since b-carotene did not produce a similar effect. UCP proteins are involved in fatty acid metabolism and their expression may be induced by certain fatty acids. It was also suggested that UCP proteins may be involved in regulation of body weight. Induced expression of UCP proteins has a potential of becoming a new promising area for the development of novel anti-obesity drugs.

Anti-obesity properties of fucoxanthin and its metabolite fucoxanthinol could also be attributed to their strong effect on adipocyte differentiation and suppression of lipid accumulation in adipose tissue via glycerol-3-phosphate dehydrogenase. In addition, fucoxanthin and fucoxanthinol downregulate peroxisome proliferator-activated receptor g (PPAR-g), which regulates adipogenic gene expression and contributes to the anti-obesity effect.

Fatty acids with conjugated double bonds have attracted considerable attention because of their potential anti-obesity effect. Conjugated linoleic acid (CLA) has been shown to reduce body fat in rodents and humans. Clinical trials showed that conjugated linoleic acid may reduce body fat mass and increase lean body mass in healthy overweight adults. Supplementation with another conjugated fatty acid, linolenic acid (CLNA) reduces adipose tissue weight in rats. It has been suggested that CLNA modulates body fat and triacylglycerol metabolism differently than CLA, although the exact mechanisms of the anti-obese action of both CLA and CLNA remain undetermined.

Punicic acid (9cis, 11trans, 13cis-conjugated linolenic acid; 9c, 11t, 13c-CLNA), a conjugated linolenic acid, is the major fatty acid found in pomegranate seed oil (23-26). Dietary pomegranate seed oil rich in punicic acid alleviates accumulation of liver triacylglycerol (TG) in obese, hyperlipidemic OLETF rats. Two weeks feeding of a diet supplemented with 5% pomegranate seed oil resulted in a significant reduction of WAT weight (by 27%) as compared with a control diet in OLETF rats, whereas feeding of a 1% pomegranate seed oil diet did not produce a significant anti-obesity effect. Thus, these results indicate that the anti-obesity effect of punicic acid is a strongly dose-dependent phenomenon. It was suggested that this effect is related to the ability of punicic acid to suppress delta-9 desaturation of fatty acid substrates in vivo, leading to decrease in hepatic TG accumulation in OLETF rats.

Therefore, punicic acid possesses properties that may indicate its usefulness in the prevention of visceral and liver fat accumulation, and as a therapeutic agent for the reduction of liver fat content in obese subjects.

Thus, there are indications that the biochemical effects of both fucoxanthin and punicic acid have an impact on the metabolic pathways responsible for deposition of visceral fat and, more specifically for NAFLD pathogenesis. Prior to this study, no human studies of the anti-obesity properties of fucoxanthin and punicic acid had been reported, and one study demonstrated effects in rodents.

Additional studies have evaluated anti-obesity effects of edible seaweed in mice and rats, with a specific focus on mitochondrial uncoupling proteins, which are responsible in part for thermogenesis and energy production. Effects of feeding Undaria pinnatifida lipids, containing a combination of glycolipids and fucoxanthin, and fucoxanthin, alone, in the diets of Wistar rats and KK-Ay (obese) mice on abdominal white adipose tissue amount, mitochondrial uncoupling protein 1 (UCP1) protein levels and mRNA levels for the protein were measured. The KK-Ay mouse is an obese-diabetic animal model, showing hyperglycemia, hypertriglyceridemia and hyperinsulinemia.

As discussed above, adipose tissue is characterized as “white adipose tissue” (WAT) or “brown adipose tissue” (BAT). Usually UCP1 is expressed only in brown adipose tissue, of which there is very little in adult humans. The hypothesis tested by the study was that increase of UCP1 expression in tissues other than BAT would be associated with a reduction in abdominal fat.

Neither mean body weight or food intake varied among rat treatment groups during the 4 week experimental period. The weights of the liver, and other organs, other than WAT, were not different among the different groups. The weight of WAT, composed of perienal and epididymal abdominal adipose tissues, was significantly lower in 2% Undaria lipid-fed rats than in the control group. In 0.5% Undaria lipid-fed rats, the weight of WAT was lower than in control, but not significantly.

There were no significant differences in the mean daily intake of diet among control and the 0.5% and 2.0% Undaria lipid-fed mice. However, in the 2% Undaria lipid-fed obese mice (KK-Ay), the weight of WAT was significantly lower than in the control group. Furthermore, body weight of mice fed 2% Undaria lipid was significantly (P<0.05) lower than that of control-fed mice. There were no differences in WAT or body weight between control and 0.5% Undaria-fed mice.

Thus, it was clear that feeding 2.0% Undaria lipids was associated with reduction of weight of WAT in rats and mice, and body weight of mice. In order to confirm the active component of Undaria lipids, fucoxanthin rich fraction and Undaria glycolipid fraction were administered to obese KK-Ay mice. The WAT weight of mice fed the fucoxanthin rich fraction was significantly lower than that of control mice. However, there was no difference in the WAT weight of mice fed Undaria glycolipids and those fed the control diet. The study concluded that this result indicated that fucoxanthin is an active component responsible for the anti-obesity effect of Undaria lipids.

To elucidate potential mechanism(s) that might contribute to the reductions of WAT and body weight, studies have also examined weight of brown adipose tissue (BAT). BAT has been implicated as an important site of thermogenesis energy expenditure in small rodents and human infants. Amount of BAT in humans decreases with aging, and adult humans have minute amounts of this energy balance regulating tissue. In animals and human infants, BAT plays a key role in thermogenesis through its capacity for uncoupled mitochondrial respiration mediated by uncoupling proteins, most notably UCP1.

Weight of BAT has been found to be significantly greater in mice fed diets containing 2% Undaria lipids. However, there was no significant difference in UCP1 expression among mice in the three dietary treatments (control, 0.5% Undaria lipid and 2% Undaria lipid). Thus, the decrease in abdominal fat pad weight of mice fed 2% Undaria lipid could not be explained by differences in UCP1 content of BAT.

An evaluation of gene expression profiles for UCP1 in WAT, the occurrence of which would suggest enhanced thermogenesis in that tissue. UCP1 expression was found in WAT of Undaria lipid-fed mice, although there was little expression in that of control mice. Expression of UCP1 mRNA was also found in WAT of Undaria lipid-fed mice, but little expression in that of control fed mice. Expression of UCP2 was also evaluated as a possible explanation of fat and weight loss in response to feeding of Undaria lipid. UCP2 is an uncoupling protein homologue associated with regulation of fatty acid vs. glucose oxidation in muscle, liver and WAT of various rodents, and in humans, is suspected of playing a role in diabetes, programmed cell death and metabolic syndrome. Further, UCP2 levels in WAT decreased in Undaria lipid-fed mice vs. controls. Accordingly, it would appear that decrease in WAT weight in Undaria lipid-fed mice was due to thermogenesis through enhanced UCP1 expression in WAT, but not due to UCP2 expression.

It has further been suggested that the seaweed carotenoid, fucoxanthin was the active component for the anti-obesity effect of Undaria lipids, and consumption of fucoxanthin at specified levels upregulated expression of UCP1 in WAT of mice. It has been hypothesized, that both fucoxanthin and punicic acid, individually or in combination, might reduce visceral fat and WAT mass in humans and have favorable effects on inflammation.

### SUMMARY

The present invention provides a method of preventing, treating or managing Metabolic Syndrome through an adjustment of one or more of the components of Metabolic Syndrome through use of an effective amount of brown marine vegetable extract standardized by fucoxanthin content. Desired, concurrent effects on visceral or central adiposity, liver enzymes elevated in NAFLD, hypertension, and/or inflammation were achieved. The amount of fucoxanthin can range from 0.5 mg to 100 mg per day, most preferably from 1.5 to 55 mg per day. Administration of effective amounts of brown marine vegetable extract standardized to those amounts of fucoxanthin has shown beneficial effects of reducing visceral adiposity, lowering blood pressure and reducing markers of inflammation as well as other beneficial effects on components of Metabolic Syndrome.

In another embodiment of the invention, brown marine vegetable extract standardized by fucoxanthin content is combined with one or more extracts or substances which provide synergistic or incremental benefits in preventing, treating or managing one or more of the symptoms of Metabolic Syndrome. For example, the combination of brown marine vegetable extract standardized by fucoxanthin content to 1.5 to 55 mg fucoxanthin per day may be combined with from 45 to 270 mg punicic acid per day, preferably 135 to 270 mg punicic acid per day, may be used.

The fucoxanthin may be administered in single daily doses or divided doses; each dose may contain from 0.5 to 55 mg fucoxanthin. Each dose may also contain from 45 to 90 mg punicic acid, preferably 70 to 90 mg punicic acid.

In another embodiment of the invention, brown marine vegetable extract standardized by fucoxanthin content to 1.5 to 55 mg fucoxanthin per day may be combined with one or more of water extract of cinnamon, chromium-containing compounds, most preferably chromium picolinate, alpha-lipoic acid, oligomeric proanthocyanidins (OPCs) such as those found in French maritime pine bark, extract of bitter melon (momordica charantia), n-3 fatty acids, eicosapentaenoic acid, n-6 fatty acids, gamma linolenic acid, and an effective amount of soluble or insoluble dietary fiber, such as glactomannans derived from Fenugreek. The fucoxanthin may be administered in single daily doses or divided doses; each dose may contain from 0.5 to 55 mg fucoxanthin.

In another embodiment of the invention, brown marine vegetable extract standardized by fucoxanthin content to 1.5 to 55 mg fucoxanthin per day is combined with one or more vitamins or minerals. The fucoxanthin may be administered in single daily doses or divided doses; each dose may contain from 0.5 to 55 mg fucoxanthin.

### DETAILED DESCRIPTION

As used herein, the term “dietary supplement” refers to compositions consumed to affect structural or functional changes in physiology.

The term “therapeutic composition” refers to any compounds administered to treat or prevent a disease.

The term “mammal” used herein refers to one selected from the group consisting of humans, non-human primates, such as dogs, cats, birds, horses, ruminants or other warm blooded animals. The invention is directed primarily, but not limited to, the treatment of human beings. Administration can be by any method available to the skilled artisan, for example, by oral, topical, transdermal, transmucosal, or parenteral routes.

As used herein, “pharmaceutically acceptable carrier” includes any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and absorbents that may be needed to prepare a particular therapeutic composition. The use of such media and agents for pharmaceutically active substances is well known in the art. Except insofar as any conventional media or agent is incompatible with the active ingredients, its use in the present composition is contemplated. Other ingredients known to affect the manufacture of this composition as a dietary bar or functional food can include flavorings, sugars, amino-sugars, proteins and/or modified starches, as well as fats and oils.

The dietary supplements or therapeutic compositions of the present invention can be formulated in any manner known by one of skill in the art. For example, the composition may be formulated into a capsule or tablet using conventional techniques with pharmaceutically acceptable excipients such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may further be coated by methods well known in the art.

However, the present compositions may also be formulated in other convenient forms such as, an injectable solution or suspension, a spray solution or suspension, a lotion, gum, lozenge, food or snack item. Food, snack, gum or lozenge items can include any ingestible ingredient, including sweeteners, flavorings, oils, starches, proteins, fruits or fruit extracts, vegetables or vegetable extracts, grains, animal fats or proteins. Thus, the present composition can be formulated into cereals, snack items such as chips, bars, gumdrops, chewable candies or slowly dissolving lozenges.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. The preparations may be suitably formulated to give controlled release of the active compounds.

The present compositions and formulations have a medicinal or health effect of a treatment of liver fat and body fat, a reduction of blood pressure, an increase of the energy expenditure rate, a reduction of inflammatory C-reactive proteins and a reduction of plasma aminotransferase enzymes.

In an exemplary study, the effect of different doses of an experimental sample had on the energy expenditure on obese subjects was reviewed. Obese subjects diagnosed with NAFLD and with apparently healthy liver (HL) were matched in pairs based on age, body weight and body fat mass and were randomly divided into Experimental NAFLD group (n=36), Placebo NAFLD (n=36), Experimental HL (n=19) and Placebo-HL group (n=19). Subjects were randomly assigned, in equal numbers, to the phytomedicine experimental groups and the Placebo control group, using the Simple Randomization Procedure. Their daily dietary intake was restricted to 1800±100 kcals, of which 50±5% was in the form of carbohydrates, 30±5% from protein, and 20±5% from fat. Subjects were also instructed to consume all the foods and beverages designated by dieticians and provided by Center of Modern Medicine, Institute of Immunopathology, Moscow and to eat no other food or high calories beverages. Patients were directed to take Experimental Sample and/or Placebo three times a day before meals. During the clinical phase, subjects were required to visit a designated hospital three times a week for physiological and biochemical analysis. The Institute provided all foods and beverages by designated dieticians and labeled as B, L, and D for breakfast, lunch and dinner, respectively.

In one aspect, the present invention provides a composition for treating metabolic syndrome by administering an effective amount of extract of brown marine vegetable. In a second aspect, the present invention provides a method of treatment for reducing inflammation. In a third aspect, the present invention provides a method for treatment of hypertension. Fucoxanthin can be obtained from marine vegetables. More particularly, the process for obtaining fucoxanthin includes the steps of: cultivating brown marine vegetables in a photobioreactor with continuous flow of circulating deep-sea water and with illumination intensity less than full sun light intensity; washing with fresh water; freeze drying the harvested marine vegetables; and extracting pharmacologically active components using supercritical CO2 fluid extraction with alcohol as co-solvent.

Preferably, the composition according to the present invention in which the brown marine vegetable extract contains fucoxanthin, fucoxanthinol and marine vegetable oil. Preferably, the brown marine vegetable extract is suspended in a solution containing at least 70% punicic acid such as cold pressed pomegranate seed oil.

Clinical studies of effects of a phytomedicine containing fucoxanthin and pomegranate seed oil, on energy expenditure rate (Example 1), and on body weight, liver fat, serum triglycerides, C-reactive protein, and plasma amino transferases (Example 2) in volunteers with and without non-alcoholic fatty liver disease. Both trials were designed as double blind, randomized placebo controlled studies. Caloric intake in both studies was 1800 kcal/day, a moderately calorie restricted diet; distribution of those calories among carbohydrate, protein and fat varied slightly between the studies. Subjects in both studies were non-diabetic, obese females with average body weight of ˜190 to 200+ lbs. Body Mass Index was reported for study 2 as >30 Kg/m2, which is consistent with a definition of obese.

### EXAMPLE 1

Effect of Fucoxanthin a phytomedicine containing fucoxanthin and pomegranate seed oil, on energy expenditure rate in obese non-diabetic female volunteers with non-alcoholic fatty liver disease: a double-blind, randomized and placebo-controlled trial.

The objectives of the study were to investigate the effects of different doses of a brown marine vegetable extract standardized by fucoxanthin content, pomegranate seed oil containing a minimum of 70% punicic acid, and the phytomedicine combining, both of fucoxanthin and pomegranate seed oil, on energy expenditure rate (EER) in obese non-diabetic female volunteers with non-alcoholic fatty liver disease (NAFLD) on a moderate calorie restriction diet.

Forty-one (n=41) volunteers with an average body weight of 91.5±14.4 kgs (201+9.7 lbs), body fat of 40.4±3.7 kgs (88.8+8 lbs), liver fat content above 10% and average age of 37.4±4.8 years were recruited to take part in a 16-week clinical trial. Daily meal composition was 50% carbohydrate, 30% protein and 20% fat. Food record data, body composition, EER, and blood sample analysis results were assessed on admission and every week for 16-weeks. Energy expenditure rate (EER) was measured by indirect calorimetry. Plasma levels of inflammatory markers alanine aminotransferase (ALT), aspartate aminotransferase (AST), g-glutamyltransferase (GGT) enzymes and C-reactive protein (CRP) were evaluated as measures of liver function, along with diastolic and systolic blood pressure, dual-energy X-ray absorptiometry to determine percent of lean and fat body mass, and liver fat content.

Subjects were randomly assigned to one of 10 treatments or placebo (total 11 groups), which were in softgel capsules and were to be consumed with water 15 to 30 mins. before meals. Each phytomedicine soft-gel capsule provided 100 mg of Undaria pinnatifida (Phaeophyceae) brown marine vegetable extract and 100 mg of cold-pressed pomegranate (Punica granatum, Punicaceae) seed oil.

The phytomedicine, fucoxanthin, pomegranate seed oil and placebo (olive oil) were well tolerated by all subjects. All subjects completed 16 weeks clinical trial without any reported adverse effects. The supplementation of both fucoxanthin and the phytomedicine increased EER in obese subjects, and the effect was dose-dependent and time-dependent. Treatment groups and effects are shown in Table 1.

TABLE 1

| | Placebo | | | | | | | | | Pomegranate | |
|---|---|---|---|---|---|---|---|---|---|---|---|
| | Olive | Phytomedicine (mg) | | | | | | | | Seed Oil (mg) | |
| Treatment | Oil | 200 | 400 | 600 | 1000 | Fucoxanthin (mg) | | | | 1500 | 2000 |
| Mg | 0 | 0.84 | 1.68 | 2.52 | 4.20 | 1.68 | 2.52 | 4.20 | 8.40 | 0 | 0 |
| Fucoxanthin/ | | | | | | | | | | | |
| day | | | | | | | | | | | |
| Effects on | NE | NE | *, 16 | *, 5 | *, 5 | NE | * | * | *** | NE | NE |
| EER | | | Weeks | Weeks | Weeks | | | | | | |

NE = No effect
* = significant vs. placebo and vs. baseline, p < 0.05% at the week listed for the duration of the study
*** = significant vs. placebo and vs. baseline, p < 0.001%

The results shown in Table 1 indicate the potential synergistic effects of fucoxanthin and pomegranate seed oil on EER. With 600 mg of the phytomedicine and 1000 mg of the other phytomedicines, the EER continued to rise until the end of 16 weeks of clinical trial, however there were no significant differences in extent of EER increase between the 2 treatments. Effects of 8.40 mg fucoxanthin on EER were highly significant vs. those of placebo and baseline and were also significant vs. those of 4.20 mg fucoxanthin. In this study, 400 mg of the phytomedicine and 2.52 mg fucoxanthin were the minimum doses that resulted in statistically significant increases in EER. Thus, those doses may approximate the minimal effective doses.

The clinical trial establishes that both fucoxanthin and the phytomedicine supplemented orally produce a statistically significant dose-dependent increase in EER in obese non-diabetic female subjects with NAFLD.

### EXAMPLE 2

The effect of Xanthigen™, a phytomedicine containing fucoxanthin and pomegranate seed oil, on body weight and liver fat, serum triglycerides, C-reactive protein, and plasma aminotransferases in obese non-diabetic female volunteers: a double-blind, randomized and placebo-controlled trial

Objectives of this study were to investigate the effect of orally administered Xanthigen™, the phytomedicine containing a combination of fucoxanthin and pomegranate seed oil, on body weight, body and liver fat content, serum triglycerides (TG), C-reactive protein (CRP), and plasma aspartate aminotransferase (AST), alanine aminotransferase (ALT), and g-glutamyltransferase (GGT) in obese, non-diabetic female volunteers with high and low liver fat content on a moderate calorie restricted diet of 1800 kcal/day. As with the previous study, CRP, AST, ALT and GGT are measures of inflammation and liver status. Dietary composition was 50% carbohydrates, 25% protein and 25% fat.

Seventy two (n=72) obese pre-menopausal female subjects with liver fat content above 11% (nonalcoholic fatty liver disease, NAFLD cluster) were randomly assigned into either Xanthigen-NAFLD group (n=36) or placebo-NAFLD group (n=36). Volunteers in this cluster had an average age of 36.7±2.5 years, average body weight of 93.8±2.2 kg (206+4.8 lbs.), body fat content 42.2±1.9 kg and plasma TG 193±17 mg/dl.

In addition, thirty eight (n=38) obese female subjects with liver fat content below 6.5% (normal liver fat, NLF cluster), were randomly divided into Xanthigen-NLF (n=19) and the placebo-NLF groups (n=19). Volunteers in this cluster had an average age of 35.2±3.2 years, average body weight of 94.2±1.8 kg (207+4 lbs.), body fat content of 43.0±1.7 kg, and plasma TG 176±12 mg/dl.

There was no statistically significant difference in the waist circumference between the groups within each cluster, but there was a statistically significant difference (p<0.05) between the clusters (smaller in the NLF cluster). Baseline values for ALT, AST, and GGT were significantly lower in NLF subjects vs those in NAFLD subjects, indicating more normal liver function in NLF subjects at the start of the study. CRP levels were not different between groups at the start of the study.

Each capsule of Xanthigen™ used in this study provided with a minimum 100 mg of brown marine vegetables extract (containing 0.84% fucoxanthin, up to 1.0% fucoxanthinol and 30 mg marine vegetable oil) that was suspended in 100 mg of cold-pressed pomegranate seed oil (containing a minimum of 70% punicic acid) equivalent to a 200 mg softgel capsule. Patients were directed to take capsules of either Xanthigen™ or placebo (olive oil) 3 times a day 15-30 minutes before each meal for 16 weeks. Thus, subjects in the Xanthigen™-treatment group consumed the minimum effective dose of fucoxanthin identified in Study 1. Daily caloric intake was restricted to 1800 kcal. Treatments are summarized in Table 2.

TABLE 2
Treatment variables for Study 2.

| | Placebo | Xanthigen |
|---|---|---|
| Treatments | Olive Oil | 200 mg softgel 3X/day |
| Mg. Fucoxanthin/day | 0 | 2.52 |

Results of the trial showed that in obese non-diabetic female subjects, Xanthigen™ supplementation for 16 weeks resulted in a statistically significant changes in several of the parameters measured. Time of on-set of significant differences vs. placebo differed between NLF and NAFLD groups.

Body weight, body and liver fat content and systolic and diastolic blood pressure, components of Metabolic Syndrome, were significantly reduced in both NLF and NAFLD Xanthigen™ treatment groups as compared to placebo. Significant weight loss and reduction in body and liver fat content were first registered in patients with NLF earlier (6 weeks) than in patients with NAFLD (8 weeks). Weight loss continued at statistically significant levels from those time points forward until the end of the 16 week study period in both groups. Xanthigen-NAFLD subjects lost ˜12.1 lbs more than those receiving placebo, of which ˜7.7 lbs. was body fat. Similar to body weight, significant reduction in % liver fat occurred at week 8 for the NAFLD group, decreasing by ˜5% by the end of the trial, which was highly significant (p<0.001 vs. placebo). The phytomedicine-NLF subjects lost ˜10.8 lbs more than those receiving placebo, of which ˜7.9 lbs was body fat. Reduction in liver fat % was evident at 5 weeks, reaching 1.7% reduction by the end of the trial, which was significant vs placebo (p<0.05).

A statistically significant decrease in waist circumference from ˜110 cm to ˜105 cm was measured at the 16 week point in subjects with NAFLD as compared to placebo (p<0.05). In the NLF cluster, there was a strong trend in waist circumference reduction in patients taking the phytomedicine; however, the effect was not statistically significant vs. that of placebo. Central or visceral adiposity, as measured by waist circumference, is a key indicator of the presence of or predisposition to Metabolic Syndrome. Thus, the phytomedicine is shown to be effective in reducing this key aspect of Metabolic Syndrome.

A statistically significant reduction in the levels of TG, AST, ALT, GGT and CRP occurred in subjects with NAFLD, in which these markers (except CRP) on admission were significantly higher than in patients with NLF. While levels of AST, ALT and GGT were reduced in NLF subjects at the end of the trial, reductions were not statistically significant. CRP levels in NLF subjects were significantly reduced at the end of the trial. Thus, the phytomedicine supplementation was shown to be effective in reduction of inflammation, as measured by CRP and liver enzymes AST, ALT and GCT.

Both NAFLD and NLF clusters showed statistically significant reductions in systolic and diastolic blood pressure at the end of the trial. Thus, the phytomedicine supplementation was shown to be highly effective in reducing blood pressure, which when elevated is another key feature of Metabolic Syndrome. Serum triglyceride levels in the phytomedicine-NAFLD group at the end of the trial were statistically significantly reduced compared to those of subjects receiving placebo (p<0.05). However, serum triglyceride levels in the phytomedicine-NLF group at the end of the trial were not statistically significantly different from those of subjects receiving placebo (p=NS).

The clinical trial provided the first human evidence of anti-obesity effects of a phytomedicine formed as a combination of a minimum 100 mg of brown marine vegetables extract (containing 0.84% fucoxanthin, up to 1.0% fucoxanthinol and 30 mg marine vegetable oil) suspended in 100 mg of cold-pressed pomegranate seed oil (containing a minimum of 70% punicic acid) equivalent to a 200 mg softgel capsule.

The use of fucoxanthin for MetS may also be accomplished by extracts of other brown marine vegetable. Ingestion of the described phytomedicine or fucoxanthin leads to weight loss, reduction in waist circumference and reductions in other biomarkers of obesity.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A composition for treating metabolic syndrome, the composition comprising:
brown marine vegetable extract containing an effective amount of fucoxanthin; and
an effective amount of punicic acid.

2. The composition of claim 1, wherein the effective amount of fucoxanthin is administered in a single dose or multiple doses, said effective amount of fucoxanthin comprising between approximately 1.5 mg to approximately 55 mg of fucoxanthin per day.

3. The composition of claim 1, wherein the composition comprises a single dose of brown marine vegetable extract having between approximately 0.5 mg to approximately 55 mg of fucoxanthin.

4. The composition of claim 1, further comprising an amount of punicic acid between approximately 45 mg and approximately 90 mg.

5. The composition of claim 4, wherein the amount of punicic acid is approximately 70 mg.

6. The composition of claim 4, wherein the punicic acid is included as a component of pomegranate seed oil.

7. The composition of claim 1, further comprising water extract of cinnamon.

8. The composition of claim 1, further comprising a chromium compound.

9. The composition of claim 8, wherein the chromium compound is chromium picolinate.

10. The composition of claim 1, further comprising at least one of an alpha-lipoic acid, an eicosapentaenoic acid, and a gamma linolenic acid.

11. The composition of claim 1, further comprising an oligomeric proanthocyanidin.

12. The composition of claim 1, further comprising at least one of an n-3 fatty acid and an n-6 fatty acid.

13. The composition of claim 1, wherein the composition is suitable for oral administration.

14. The composition of claim 1, wherein the composition is suitable for intravenous administration.

15. The composition of claim 1, wherein the composition is suitable for topical administration.

16. A method for treating metabolic syndrome, comprising the step of:
administering a composition comprising brown marine vegetable extract having between approximately 0.5 mg to approximately 100 mg of fucoxanthin.

17. The method of claim 16, further comprising the step of assessing a condition of at least one of obesity, glucose intolerance, and hypertension.

18. The method of claim 16, wherein the step of administering the brown marine vegetable extract includes administering between approximately 1.5 mg and approximately 55 mg of fucoxanthin per day.

19. The method of claim 16, wherein the step of administering the brown marine vegetable extract includes administering between approximately 0.5 mg to approximately 55 mg of fucoxanthin per dose.

20. The method of claim 16, wherein the composition further includes an amount of punicic acid between approximately 45 mg and approximately 90 mg.

21. The method of claim 20, wherein the amount of punicic acid is approximately 70 mg.

22. The method of claim 20, wherein the punicic acid is pomegranate seed oil.

23. The method of claim 16, wherein the composition further includes water extract of cinnamon.

24. The method of claim 16, wherein the composition further includes a chromium compound.

25. The method of claim 24, wherein the chromium compound is chromium picolinate.

26. The method of claim 16, wherein the composition further includes at least one of an alpha-lipoic acid, an eicosapentaenoic acid, and a gamma linolenic acid.

27. The method of claim 16, wherein the composition further includes an oligomeric proanthocyanidin.

28. The method of claim 16, wherein the composition further includes at least one of an n-3 fatty acid and an n-6 fatty acid.

29. A composition for treating metabolic syndrome, the composition comprising:
brown marine vegetable extract in an amount containing an effective amount of fucoxanthin; and
an effective amount of pomegranate seed oil.

30. The composition of claim 29, further comprising an additive selected from the group consisting of:
a water extract of cinnamon,
a chromium-containing compound,
alpha-lipoic acid,
oligomeric proanthocyanidins,
extract of bitter melon (momordica charantia),
n-3 fatty acids,
n-6 fatty acids,
an effective amount of soluble or insoluble dietary fiber, and combinations thereof.
